(19) United States
(12) Patent Application Publication
(10) Pub. No.: US 2022/0025631 A1
(43) Pub. Date: Jan. 27, 2022

(54) CONTAMINANT TYPE DETECTION SYSTEM AND METHOD

(71) Applicant: ECZACIBASI YAPI GEREÇLERI SANAYI VE TICARET ANONIM SIRKETI, Istanbul (TR)

(72) Inventors: Aykut KIZICI, Bozuyuk, Bilecik (TR); Fatih GERENLI, Istanbul (TR); Bogac SIMSIR, Istanbul (TR)

(21) Appl. No.: 17/311,435

(22) PCT Filed: Dec. 16, 2019
(86) PCT No.: PCT/TR2019/051085

§ 371 Date: Jun. 7, 2021

(51) Int. Cl. E03D 11/13; A61B 10/00; G06F 16/23; H04W 4/38

(52) U.S. Cl. CPC E03D 11/13; H04W 4/38; G06F 16/2358; A61B 10/007

(57) ABSTRACT

A contaminant type detection system and method, which enable detection of the type of the contaminants contained by wireless signals, before or after the use, by the liquid within the component of the ceramic sanitary ware that provides the connection of the ceramic sanitary ware with the wastewater installation, in other words, within the elbow structure, and which, in case a contaminant other than the expected is encountered, enable to inform the user and/or third parties about the contaminant.

### SUBJECT OF THE INVENTION

The invention relates to a contaminant type detection system and method, wherein the type of the contaminant accumulating after use within a toilet bowl or squatting pan as a result of the use of said toilet bowl or squatting pan is detected and in case an unexpected contaminant is encountered or in case the liquid present within the toilet bowl or squatting pan prior to use is different from the reference clean water values, the third parties are informed depending on the preferences and the needs of the user.

### STATE OF THE ART

The toilet bowls and squatting pans are used in the bathrooms and toilet rooms in order for the persons to excrete their bodily wastes from their excretory system and for said wastes to be conveyed to the wastewater installation. Some quantity of liquid is always present within the elbow structure of the toilet bowls or squatting pans and this liquid becomes contaminated after the use of the toilet bowl or squatting pan, in other words, after the excretion.

In the practices according to the state of the art, most of the time the pollution level of the liquid within the elbow structure is attempted to be detected, and the flushing system is intended to be automatically activated according to the pollution level.

The wastes of the human excretory system give many hints about the health state of a person. In fact, the urine analyses are utilized in the diagnosis of many diseases. However, the urine analysis is performed mostly as a result of a person applying to a hospital for certain complaints. On the other hand, in case this analysis is performed regularly at certain intervals, it could be possible to detect some health problems even before the occurrence of the symptoms. Thus, the toilets, which are used almost every day for the excretion of the bodily wastes from the excretory system, are suitable environments for regular analyses of urine and stool. The toilets developed for this purpose are available in the state of the art. In an exemplary embodiment according to the state of the art, a tube emerges from within the toilet bowl after the use, takes a urine sample and measures the quantity of glucose within the urine to determine the blood sugar. In such and other similar embodiments, the necessity to take a sample for analysis makes the operation of the system difficult and causes the system to become a complex structure.

In some other cases, the liquid, which is present prior to use within the toilet bowl or squatting pan and which is expected to be clean, is observed to be not sufficiently clean as a result of being contaminated by various contaminants before the use for various reasons. The detection of such a situation is most of the time impossible in cases where such contaminants are not of the odor-emitting type.

In the contaminant type detection system and method according to the invention, the excretory system wastes within the contaminated liquid are analyzed prior to activating the flushing system after the use, without having to take a sample from the contaminated liquid within the toilet bowl or squatting pan, and in case an unexpected contaminant is encountered within the wastes and/or in case the wastes have caused an unexpected effect and/or in case the values of the liquid present within the toilet bowl or squatting pan prior to use are different from the expected values, in other words, from the reference clean water values, the third parties are enabled to be informed depending on the preferences and the needs of the user.

### OBJECT OF THE INVENTION

An object of the invention is to develop a contaminant type detection system and method, which enable to detect the type of the contaminant within the elbow structure of a toilet bowl or squatting pan following the use of said toilet bowl or squatting pan.

Another object of the invention is to develop a contaminant type detection system and method, wherein in case an unexpected contaminant is encountered within the elbow structure of a toilet bowl or squatting pan following the use of said toilet bowl or squatting pan, the user and/or the third parties are informed depending on the preferences and the needs.

Another object of the invention is to develop a contaminant type detection system and method, wherein in case the values of the water present within the toilet bowl or squatting pan prior to use are different from the predefined reference clean water values, the user and/or the third parties are informed depending on the preferences and the needs.

### DESCRIPTION OF THE FIGURES

FIG. 1: A side sectional view of an embodiment where the transmitter/receiver is positioned perpendicular to the floor.

FIG. 2: A front sectional view of an embodiment where the transmitter/receiver is positioned perpendicular to the floor.

FIG. 3: A perspective view of an embodiment where the transmitter/receiver is positioned perpendicular to the floor.

FIG. 4: A sectional view of an embodiment where the transmitter/receiver is positioned perpendicular to the floor.

FIG. 5: A bottom view of an embodiment where the transmitter/receiver is positioned parallel to the floor.

FIG. 6: A side sectional view of an embodiment where the transmitter/receiver is positioned parallel to the floor.

FIG. 7: A bottom view of an embodiment where the transmitter/receiver is positioned parallel to the floor.

FIG. 8: A front sectional view of an embodiment where the transmitter/receiver is positioned parallel to the floor.

FIG. 9: An angled rear view of an embodiment where the transmitter/receiver is positioned parallel to the floor.

FIG. 10: An angled bottom view of an embodiment where the transmitter/receiver is positioned parallel to the floor.

FIG. 11: An angled side view of an embodiment where the transmitter/receiver is positioned parallel to the floor.

FIG. 12: A graph showing the amplitude variations for three different samples.

FIG. 13: A graph comparing the amplitude variations for three different samples.

FIG. 14: A graph showing the phase variations for three different samples.

FIG. 15: A graph comparing the phase variations for three different samples.

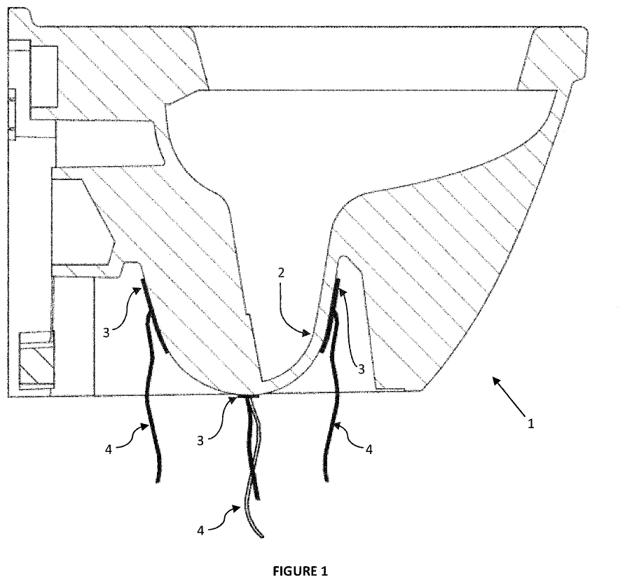

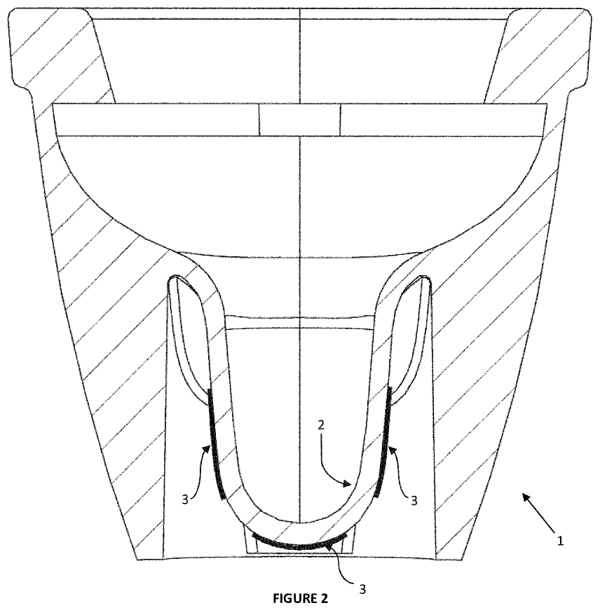

### REFERENCE NUMERALS

1: Ceramic sanitary ware
2: Elbow structure
3: Transmitter/receiver
4: Transmitter/receiver connection

### DETAILED DESCRIPTION OF THE INVENTION

The invention relates to a contaminant type detection system and method, wherein the type of the contaminant accumulating after use within a toilet bowl or squatting pan as a result of the use of said toilet bowl or squatting pan is detected and in case an unexpected contaminant is encountered or in case the liquid present within the toilet bowl or squatting pan prior to use is different from the reference clean water values, the third parties are informed depending on the preferences and the needs of the user. More particularly, the invention relates to a contaminant type detection system and method, which enable to detect the type of the contaminants contained before or after the use by means of wireless signals, by the liquid within the component of the ceramic sanitary ware (1) that provides the connection of said ceramic sanitary ware (1) with the wastewater installation, in other words, within the elbow structure (2), and which, in case a contaminant other than the expected is encountered, enable to inform the user and/or third parties about said contaminant. The ceramic sanitary ware (1) may be a urinal, a Turkish style toilet and a toilet bowl, but it is not limited thereto and may be in the form of any ceramic sanitary ware (1).

In the contaminant type detection system and method according to the invention, at least one transmitter/receiver (3), which is positioned in a way to see the elbow structure (2) and/or which is positioned on the elbow structure (2), is utilized in order to detect the contaminant type. The wireless signals sent by the transmitter/receiver (3) pass through the liquid present within the elbow structure (2) and reach the same transmitter/receiver (3) or a different transmitter/receiver (3). More specifically, the signals sent by a first transmitter/receiver (3) may pass through the elbow structure (2) and/or the liquid within the elbow structure (2) and may be caught by a second transmitter/receiver (3). Depending on the variation in the wireless signals, the contaminant type contained by the liquid within the elbow structure (2) is determined.

The number and positions of the transmitter/receiver s (3) being employed may vary depending on the preferences and needs of the user. The transmitter/receivers (3) are preferably positioned in a way to face the elbow structure (2) or positioned on the elbow structure (2) on the same level as the liquid within the elbow structure (2). In the embodiments where it is preferred to use a single transmitter/receiver (3), the signals sent by the transmitter/receiver (3) reach the contaminated liquid within the elbow structure (2). The electromagnetic properties, i.e. dielectric properties, of the liquid vary according to the types and quantities of contaminants present within the liquid and the signals that are reflected to return to the transmitter/receiver (3) accordingly vary depending on the dielectric properties of the liquid. The contaminants contained by the liquid may be determined based on such differences.

In a preferred embodiment of the invention, more than one transmitter/receiver (3) is used. Again, these transmitter/receivers (3) are positioned on the elbow structure (2) or positioned in a way to face the elbow structure (2), such that one transmitter/receiver (3) will be disposed opposite at least one other transmitter/receiver (3). According to this embodiment, the signals sent by the transmitter/receiver (3) acting as the transmitter pass through the elbow structure (2) and the liquid within the elbow structure (2) and are received by another transmitter/receiver (3), which is positioned opposite first transmitter/receiver (3) and which acts as the receiver. Depending on the difference between the signal sent by the transmitter/receiver (3) acting as the transmitter and the signals received by the transmitter/receiver (3) acting as the receiver, the contaminants contained by the liquid within the elbow structure (2) may be determined.

In another preferred embodiment of the invention, one or more transmitter/receiver (3) operating based on the principle of a transmitter/receiver (3) receiving the signals sent also by the same transmitter/receiver (3) and two or more transmitter/receiver s (3) operating based on the principle of a transmitter/receiver (3) receiving the signals sent by another transmitter/receiver (3) may be used in combinations that include different numbers of such transmitter/receivers (3).

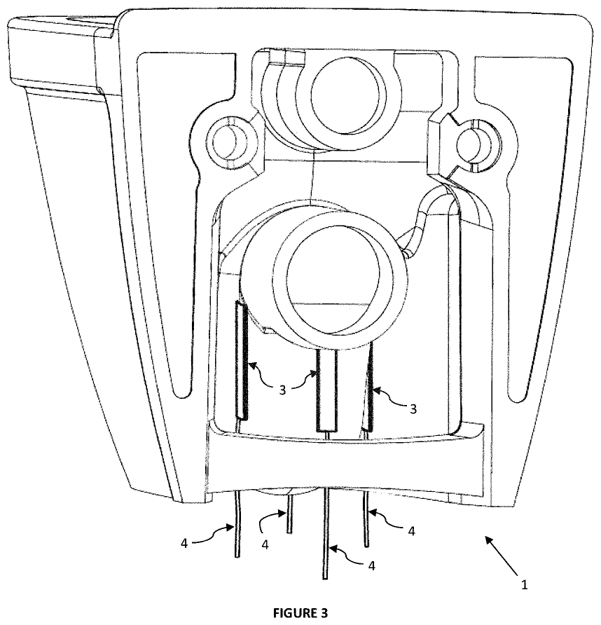

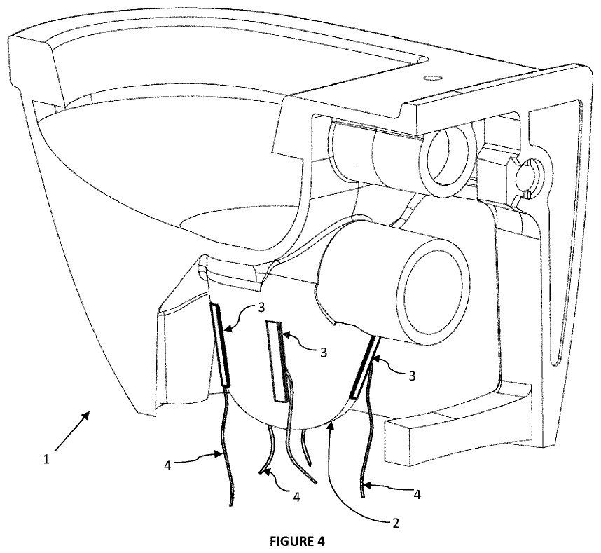

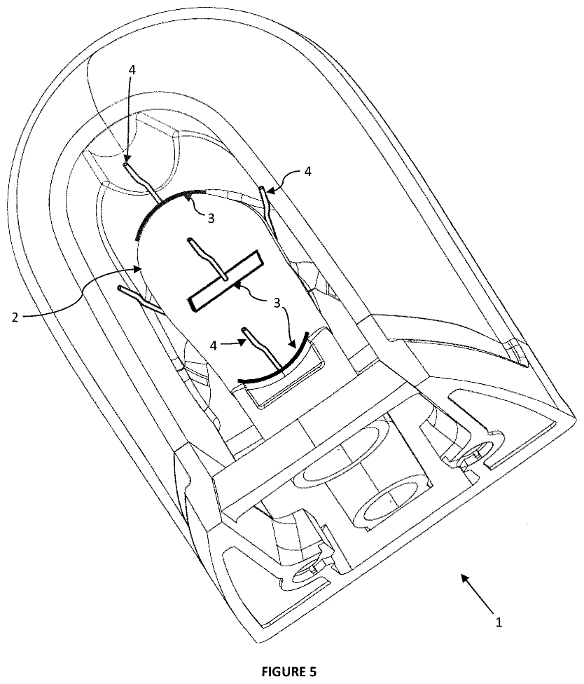

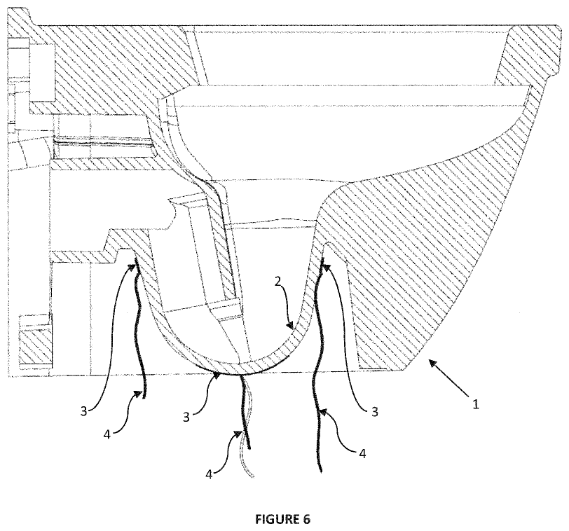

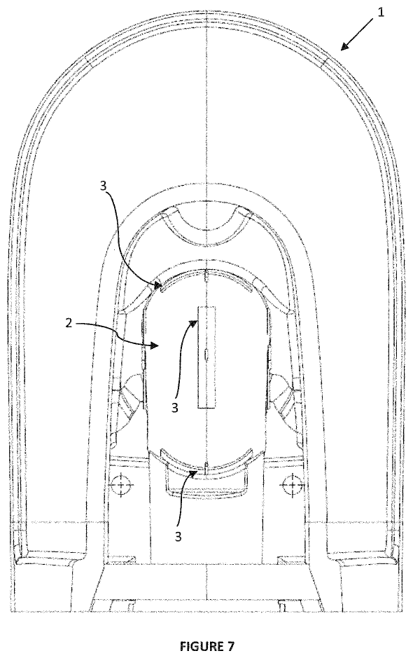

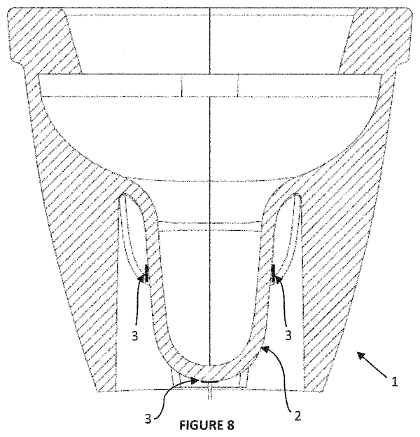

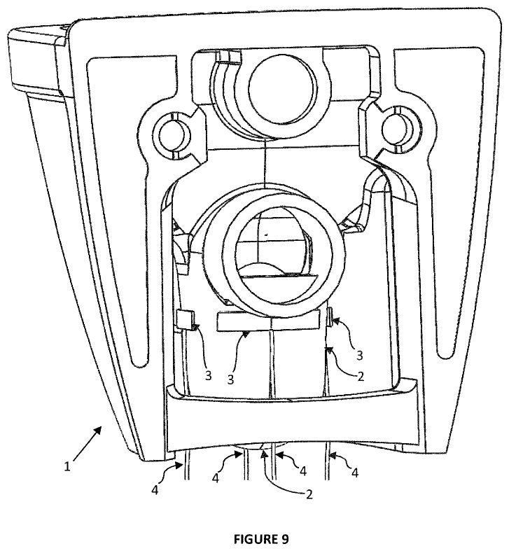

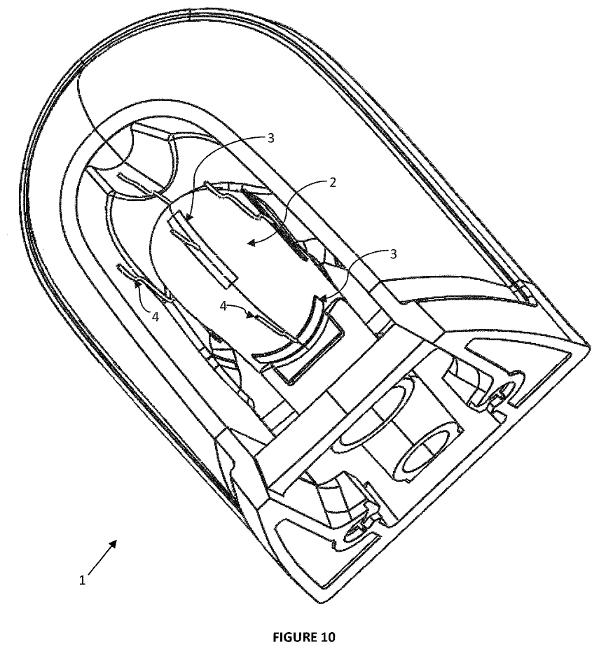

Depending on the needs, preferences and area of use, the transmitter/receiver s (3) may be positioned anywhere in a way to face the elbow structure (2) and/or on the elbow structure (2), such that they will send signals in a direction parallel to the floor or in a direction perpendicular to the floor. In other words, the transmitter/receiver s (3) may be positioned on the elbow structure (2) in a way to extend upwards from the floor as shown in FIG. 3, while it is also possible for the same to be positioned in a direction parallel to the floor and/or in an angled manner with respect to the floor.

A preferred embodiment of the invention comprises at least one main processing unit. The transmitter/receivers (3) are preferably connected to the main processing unit by means of a transmitter/receiver connection (4). The main processing unit enables a transmitter/receiver (3) to send a signal, the sent signal to be received by another transmitter/receiver (3) or the reflections to be received by the same transmitter/receiver (3). In a preferred embodiment of the invention, the transmitter/receiver (3) operates in the band range of 1 kHz-6 GHz. In another preferred embodiment of the invention, the transmitter/receiver (3) operates in the band range of 6 GHz-18 GHz. In another preferred embodiment of the invention, the transmitter/receiver (3) operates in the band range of 18 GHz-40 GHz. In another preferred embodiment of the invention, the transmitter/receiver (3) operates in the band range of 40 GHz-100 GHz. The received signals are recorded preferably as amplitude and/or phase and said recorded amplitude and/or phase are compared to the predefined reference values in order to determine the contaminant type within the elbow structure (2).

Each different contaminant present within the elbow structure (2) of the ceramic sanitary ware (1) and the quantities of such contaminants cause different variations in the dielectric value of the liquid; in other words, they cause different variations in the amplitude and/or phase of the signals detected by the transmitter/receiver (3). The variations in the amplitude and/or phase caused by different contaminant types are predefined in the main processing unit and the amplitude and/or phase values of the liquid within the elbow structure (2) before the use and/or after the use are compared to these reference values, and in this way, the types and quantities of the contaminants present within the liquid may be detected. Depending on the choices and preferences of the user and/or manufacturer, different procedures may be implemented and different persons may be informed according to different contaminant types.

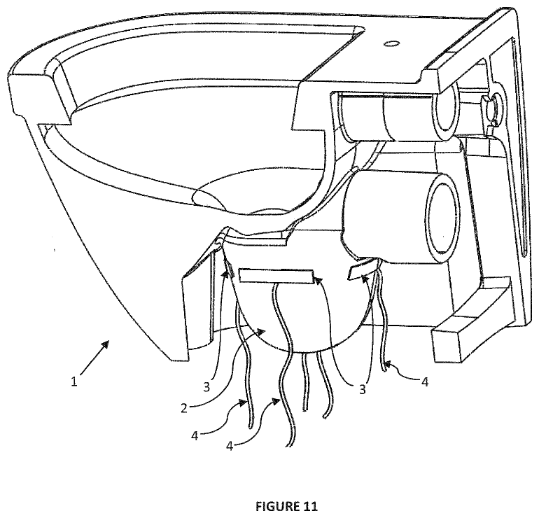

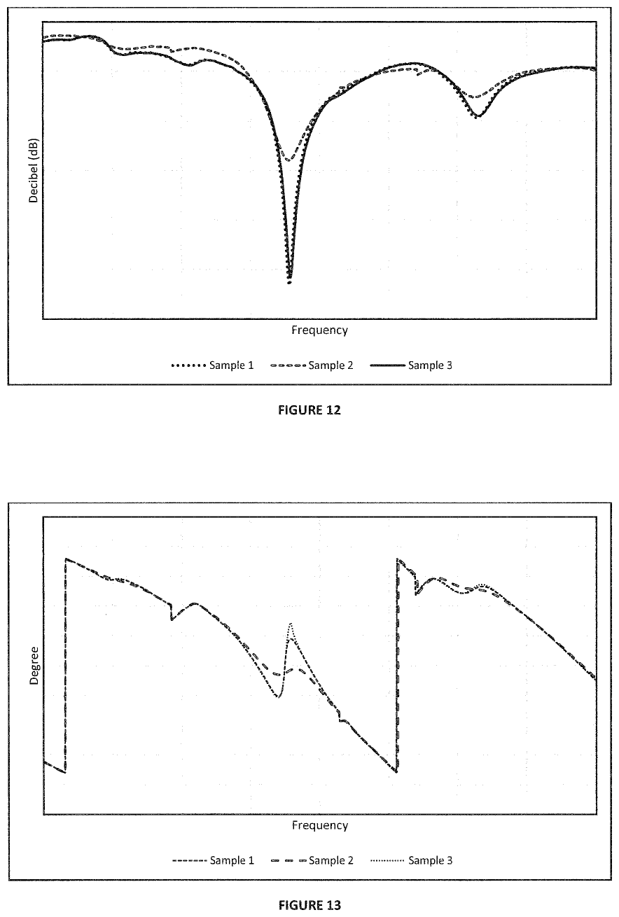

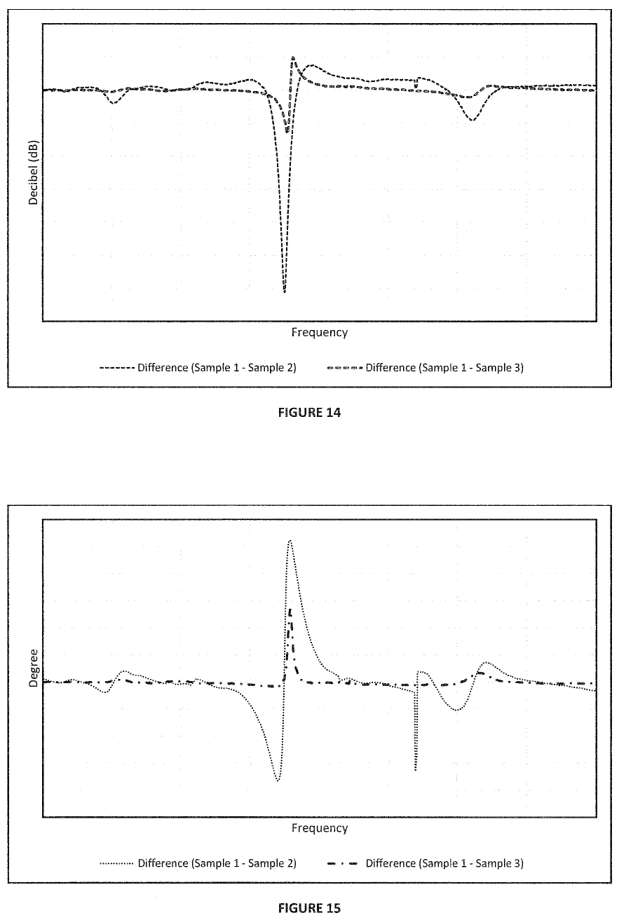

FIG. 12 shows the amplitude values for three different samples and FIG. 13 shows the amplitude comparison between Sample 1 and Sample 2 and between Sample 1 and Sample 3. Similarly, FIG. 14 shows the phase values for three different samples and FIG. 15 shows the phase comparison between Sample 1 and Sample 2 and between Sample 1 and Sample 3. It may be seen in the figures that different samples have different amplitude and phase values.

The variations caused by the contaminants and their quantities in the amplitude and phase values exhibit differences. The type of contaminant and the quantity of contaminant may be detected based on such differences.

In case blood or a similar biological component, which is not expected to be present under normal conditions within the excretory system waste, is encountered within the elbow structure (2) of the ceramic sanitary ware (1), the user and/or the third parties that the user prefers to be informed are provided with information that an unexpected contaminant has been detected and the contaminant is a contaminant originating from the user. Such information may be provided via an internal display and/or via a notification sent to any device, with network connection, of the user and the third party preferred to be informed by the user. In a preferred embodiment of the invention, the unexpected contaminants contained by the excretory system waste may be recorded and stored in a database. Such information included in the database may be utilized in order to track certain biological data of a person over the long term. In another preferred embodiment of the invention, the start and end dates for the menstrual cycle of a person may be automatically determined and recorded, wherein the person may be warned about the duration of the cycle, about any delay in the cycle or about any early start for the cycle and/or may be informed about the approximate start date for the next cycle. Similarly, the predictions may also be made about the pregnancy by way of evaluating, separately or together, the delay in the menstrual cycle and the variations caused by the contaminant in the amplitude and/or phase of the wireless signals.

In a preferred embodiment of the invention, the types and quantities of contaminants contained by the water that is present within the elbow structure (2) prior to use may also be determined. The values for the clean water are previously recorded in the system as the reference values, the values of the liquid within the elbow structure (2) prior to use are compared to the predefined reference values and the presence of the contaminant within the liquid, which is expected to be clean, is evaluated based on the differences. In case the presence of a contaminant is detected within the liquid as a result of evaluation, this situation may, depending on the choices and preferences, be recorded as a contaminant that is carried with the liquid and/or that is present in the ceramic sanitary ware (1) and/or the user and/or the third parties identified by the user may be informed about the situation. The data for different ceramic sanitary ware (1) may be saved in a common database, the analysis of water may be conducted on regional basis and in case the water in a region is contaminated by the same contaminants, the authorities may be informed about the same.

In the embodiment where it is preferred to inform the user about the contaminant types via an internal display, the invention comprises at least one display operating in connection with the main processing unit. In the embodiment where it is preferred to inform the user by sending a notification and/or store the data in a database, the invention comprises at least one communication unit and the network connection is provided owing to said communication unit.

The contaminant type detection method according to the invention comprise the following process steps:
The wireless signals are sent at certain intervals by the transmitter/receiver (3) to the liquid within the elbow structure (2) and said sent signals are received by the same transmitter/receiver (3) and/or another transmitter/receiver (3) depending on the use.
The received signals are compared to the predefined reference contaminant values and the contaminant types and contaminant quantities contained by the liquid within the elbow structure (2) are detected as a result of said comparison.
In case a contaminant other than the expected is detected, depending on the choices and preferences, the user and/or the third parties identified by the user are informed about the contaminant and/or this information is saved in a database.

The system and method according to the invention will respectively become a frequently preferred system and method owing to the facts that the contaminants contained by the liquid present before and/or after the use within the elbow structure (2) are detected by means of the wireless signals without having to make any change in the structure of the existing ceramic sanitary ware (1) and without any physical contact occurring with the liquid, and that the user and/or the predefined third parties are informed about the presence and quantity of the contaminant and/or such data are collected in a database, and as a result, it would be possible to continuously track certain information pertaining to the health state of the user or to inform the user in the first step in case of a problem in the health state and to detect the common contaminants encountered in a region.

1. A contaminant type detection system, which enables detection of the contaminant types contained by a liquid within an elbow structure enabling a ceramic sanitary ware to be connected to a wastewater installation, the system comprising at least one transmitter/receiver being positioned in a way to face the elbow structure and/or being positioned on the elbow structure and enabling to detection of dielectric properties of the liquid within the elbow structure in the form of phase and/or amplitude by sending wireless signals in a way to pass through the liquid within the elbow structure.

2. A contaminant type detection system according to claim 1, wherein said transmitter/receiver is a transmitter/receiver capable of sending wireless signals in order to detect the contaminant type and capable of catching signals that pass through and are reflected from the elbow structure and/or the liquid within the elbow structure.

3. A contaminant type detection system according to claim 1, comprising at least one first transmitter/receiver capable of sending wireless signals in order to detect the contaminant type and at least one second transmitter/receiver capable of catching the wireless signals, which are sent by the first transmitter/receiver and which pass through the elbow structure and/or the liquid within the elbow structure.

4. A contaminant type detection system according to claim 1, comprising a main processing unit, which determines the signal to be sent by the transmitter/receiver, records the received signal and enables the determination of the contaminant type based on the difference between the sent and received signals.

5. A contaminant type detection system according to claim 4, wherein the that the main processing unit is a main processing unit, which records the signals sent and received by the transmitter/receiver in the form of amplitude and/or phase.

6. A contaminant type detection system according to claim 1, wherein the transmitter/receiver is a transmitter/receiver operating in the band range of 1 kHz-6 GHz.

7. A contaminant type detection system according to claim 1, wherein the transmitter/receiver is a transmitter/receiver operating in the band range of 6 GHz-18 GHz.

8. A contaminant type detection system according to claim 1, wherein the transmitter/receiver is a transmitter/receiver operating in the band range of 18 GHz-40 GHz.

9. A contaminant type detection system according to claim 1, wherein the transmitter/receiver is a transmitter/receiver operating in the band range of 40 GHz-100 GHz.

10. A contaminant type detection system according to claim 1, comprising at least one display, which enables the user to be informed about the detected contaminant in case an unexpected contaminant is detected in the liquid within the elbow structure.

11. A contaminant type detection system according to claim 1, comprising at least one communication unit, which provides a network connection necessary for informing the user and/or the third parties identified by the user about the detected contaminant and for transmitting the situation information to a database, in case an unexpected contaminant is detected in the liquid within the elbow structure.

12. A contaminant type detection system according to claim 1, wherein the transmitter/receiver is positioned in a way to face the wall of the elbow structure, which enables the ceramic sanitary ware to be connected to the wastewater installation, and/or on the elbow structure, such that it is arranged parallel and/or perpendicular to the floor and/or it is arranged in an angled manner with respect to the floor.

13. A contaminant type detection method, which enables detection of contaminant types contained by a liquid within an elbow structure enabling ceramic sanitary ware to be connected to the wastewater installation, the method comprising the steps of:
sending of wireless signals at certain intervals by a transmitter/receiver to the liquid within the elbow structure and receipt of the sent signals by the same transmitter/receiver and/or another transmitter/receiver (3) depending on use;
comparing the received signals to predefined reference contaminant values and detecting contaminant types and contaminant quantities contained by the liquid within the elbow structure as a result of said comparison; and
informing a user and/or third parties identified by the user about the contaminant and/or saving this information in a database, depending on the choices and preferences, in case a contaminant other than the expected is detected.

14. A contaminant type detection method according to claim 13, wherein in case a contaminant other than the expected is detected in the liquid inside the elbow structure before the ceramic sanitary ware is used by the user, this contaminant is notified and/or saved in the database as a contaminant that is carried with the liquid or that is present in the ceramic sanitary ware.

15. A contaminant type detection method according to claim 13, wherein in case a contaminant other than the expected is encountered in the liquid inside the elbow structure after the ceramic sanitary ware is used by the user, this contaminant is notified and/or saved in the database as a contaminant that originates from the user.

16. A contaminant type detection method according to claim 14, wherein in case a contaminant other than the expected is detected, the user is informed via a display operating in connection with the main processing unit.

17. A contaminant type detection method according to claim 13, wherein in case a contaminant other than the expected is detected, a notification is sent to any device; of the user and/or the persons identified by the user with network connection.

18. A contaminant type detection method according to claim 13, wherein the start and end dates for the menstrual cycle of a user are recorded based on the detection of blood in the liquid inside the elbow structure.

19. A contaminant type detection method according to claim 18, wherein the user is informed about the possibility of pregnancy by way of evaluating, separately or together, the delay in the person's menstrual cycle and/or the variations caused by the contaminant in the amplitude and/or phase of the wireless signals.
